(19) United States
(12) Patent Application Publication
(10) Pub. No.: US 2004/0254640 A1
(43) Pub. Date: Dec. 16, 2004

(54) Needle punched textile for use in growing anatomical elements

(75) Inventors: Fraser W. H. Sutherland, Boston, MA (US); Tjorvi E. Perry, Dedham, MA (US); Srinath P. Tupil, Chelmsford, MA (US)

Correspondence Address: Robert K. Tendler, 65 Atlantic Avenue, Boston, MA, 02110, US

(73) Assignee: Children's Medical Center Corporation

(21) Appl. No.: 10/337,191

(22) Filed: Jan. 6, 2003

Related U.S. Application Data
(60) Provisional application No. 60/360,956, filed on Mar. 1, 2002.

(51) Int. Cl. A61F 2/24

(52) U.S. Cl. 623/2.13; 623/918; 435/399

(57) ABSTRACT

In a system for generating tissue by growing cells in a porous and sometimes biodegradable material, a needle punched textile which serves as a scaffold is used for growing any of a variety of anatomical elements, in which the thickness of areas of the anatomical element and thus its strength can be increased by providing layers of mesh which are needled together to form a layerless textile and in which delamination is prevented through the use of the needling. In one embodiment, the needle punched textile is utilized to form a semi-lunar heart valve. In a preferred embodiment for pediatric use, the textile is made from two different biodegradable non-woven meshes. For some adult applications biodegradable meshes are not necessary, thus eliminating the necessity of using two different needled meshes. In one embodiment the needling is done with a single needle which is made to move around the periphery of a mold used in making the scaffold, thus to precisely control the area needled. Also a method for culturing cell-scaffold construction is described.

### FIELD OF THE INVENTION

This invention relates to tissue engineered structures and more particularly to the utilization of needling to provide improved structures on which tissue may be grown.

### BACKGROUND OF THE INVENTION

The need for a substitute pulmonary valve for children is demonstrated by the fact that in the UK alone there were 488 pulmonary valve procedures performed on children (under the age of sixteen) 1999-2000 (Society of Cardiothoracic Surgeons of UK and Ireland, annual report) This figure represents the need in a population of approximately 60 million people. The population in the developed world, where there is access to pediatric heart surgery that is, in the U.S., Asia, Japan and Australia approaches 910 million. Thus it will be seen that there is substantial need for pediatric pulmonary valves.

The present device of choice is a homograft, prepared from cadavers. The fate of, homografts is however, poor with almost 70% of children requiring re-operation form replacement of faulty valves within 15 years. As most children are operated on in their early years of life, it is clear that most of these children will have their second open heart procedure before graduating from high school. The fate of small homografts and of homografts placed at second or sub sequent operations is much inferior to the 70% cited above (Stark J., Bull C., Stajevic M., Jothi M., Elliott M., de Leval M. Fate of subpulmonary homograft conduits: Determinants of late homograft failure. Journal of Thoracic and Cardiovascular Surgery 1998;115:506-16). There is furthermore, a shortage of appropriately sized homografts in Europe and the U.S. In some countries, especially in Asia, the use of preserved body parts is frowned upon for religious or cultural reasons.

In the field of pediatrics, there is also a requirement that as the child grows so do various of its anatomical elements such as the heart, the great arteries, the entire vasculature and all viscera and organs. When a pediatric patient presents with anatomical anomalies or deficiencies substituting a wholly artificial element for the deficiency is not desired because as the child grow so must these elements.

For instance, in the repair of a pulmonary valve in a neonate it is inappropriate to implant either an adult valve due to its size and the shortage of space in the infant's chest cavity, or to implant a pediatric smaller valve, if such existed, because the latter would not accommodate the growth of the child and would rapidly become flow limiting by virtue of its small size, the flow through a tube being proportional to the square of the diameter of said tube. The result is that multiple open-heart surgeries are necessary during the growth of the child.

In order to accommodate pediatric growth, in the past tissue engineered elements have been sought in which tissue is grown over a so called scaffold which is biodegradable. Upon degradation what is left is tissue which has the form of the underlying scaffold but which is thought to increase in size as the child grows.

Biodegradable polymers which have been used for tissue engineering applications are mainly based on clinically established medical devices and implants. In the group of macromolecules of natural origin collagen, alginate, agarose, hyaluronic acid derivatives, chitosan, an fibrin glue have been used as scaffolds. Man-made polymers such as polyglycolide (PGA), polylactides (PLLA, PDLA), poly(caprolactone) (PCL), and poly(dioxanone) (PDS) have also been studied as matrix materials to guide the differentiation and proliferation of cells into the targeted functional premature and/or mature tissue. Appropriate selection of scaffold material in terms of its mechanical properties and degradation characteristics with respect to the targeted tissue is essential.

Previously, biodegradable heart valve scaffolds for tissue engineering have been assembled from flat sheets of non-woven poly-glycolic acid, or PGA, fibers, impregnated with the thermoplastic polymer, poly-4-hydroxybutyrate, or P4HB. These flat sheets were then assembled into a trileaflet structure by a series of two or more wraps around a cylindrical mandrel. Seams were heat welded at a temperature above the melting point of the P4HB. Commissures could be further reinforced by three interrupted ‘suspending’ sutures.

Non-woven textiles are peculiarly well suited to tissue engineering applications as they can be made highly porous and exhibit extensive interporous connections that allow cells to penetrate and adhere to fibers through the full thickness of the scaffold. Furthermore, the fibers are not stretched taught as in a woven material therefore the material exhibits a degree of ‘give’ that, it is hoped, should not restrict subsequent growth of the nascent tissue.

However, it is noted that with the prior art there is a tendency for delamination to occur between the inner layer comprising the valve leaflets and the outer layer of the supporting conduit. There is also little support at the commissures where maximal stress is experienced by the valve leaflets in vivo. The wrap approach also requires that one valve leaflet comprise two layers of textile at the overlapping join and is therefore double the thickness. Stiffness of a sheet of material varies as the third power of its thickness. It follows, therefore, that a material that is twice as thick will be 2 to the power of 3 or 8 times as stiff. Flexibility of the valve leaflets is essential for their function in a heart valve so an unusually stiff leaflet will not function properly as the other leaflets do. Finally, the geometry of the conduit wall is seen to be that of a simple cylinder whereas the normal semi-lunar heart valve is a complex shape incorporating bulges known as sinuses of Valsalva. The presence of valve sinuses is known to be important for opening and closure of the valve leaflets during the normal function of the valve. They are also, important for stress distribution between the leaflets and conduit wall which is important for the long term structural integrity and function of the valve. The absence of sinuses of Valsalva from the prior art is therefore expected to compromise function of the valve from the moment it is implanted and to significantly decrease the longevity of any such tissue engineered heart valve.

During implantation of such valves the following problems were encountered:

1. Delamination of the scaffold with the leaflets coming away from the conduit wall

2. Spontaneous rupture of conduit wall after weaning the animal from the heart-lung machine and restoration of the circulation

3. Suture line dehiscence with sutures pulling out from the material

4. Problems imposed by overlap

5. Problems with the geometry of the conduit wall

As to delamination, in animal studies, delamination was found to be a problem at the time of implantation, that is after approximately 4 weeks of culture in the laboratory, with valve leaflets separating from the conduit wall. In the extreme; this separation resembles the pathological separation of aortic wall structures seen in congenital sinus of Valsalva aneurysms with resulting aorto-ventricular communication. Even before seeding cells onto the scaffold, it was found that layers could be separated fairly easily by hand. This finding is perhaps not surprising given that bonding between layers is provided only by contact attachment of P4HB with individual PGA fibers on the surface of each layer.

The creation of a heart valve scaffold from essentially flat sheets of mesh mandates a structure with at least two layers in its assembly. Delamination is a potential problem intrinsic to all layered structures. In general, however, it can be overcome by stitching or placement of through thickness fibers. In fact, it is known that the number of through thickness fibers required to resist delamination is only a few percent of total fiber volume within a given textile. An early solution to this problem was therefore to place individual sutures through the three commissures and a running suture around the line of attachment of the leaflet to conduit wall. However, individual sutures placed in this way are required to bear the entire load of the fabric and therefore generate stress foci. Long term failure of biological valves is highly related to stress foci and the use of sutures in biological valves have been points of failure in the past and recognized as a design failure of such prostheses.

As to spontaneous rupture and suture line dehiscence, tearing of the construct at the suture line and spontaneous rupture of the conduit portion of the tissue engineered construct was found to be a major problem at the time of surgery. Furthermore, it proved impossible to suture or repair these defects without causing further damage to the tissue, engineered structure. It was clear that greater strength would be required to maintain integrity of the construct at the proposed time of implantation, after 4 weeks of culture in the laboratory, and beyond. The strength of the construct derives from a combination of extra cellular matrix laid down by cells during the conditioning period and the tensile properties of the underlying scaffold.

As to problems imposed by overlap, the simplest means of assembling a cylindrical structure from a flat sheet is by way of a wrap with an overlapping join. One type of heart valve scaffold is assembled from two such wraps. Whilst this method is attractive in its simplicity, the presence of overlapping joins creates an asymmetrical structure with two specific problems. Firstly, part of one leaflet is necessarily two layers thick. Thickened leaflets are a problem in general, but symmetry, affects movement and stress/strain within that leaflet with long-term implications. Secondly, there is unequal support offered to the leaflets at the three commissures owing to the overlap.

As to the geometry of the conduit wall, setting aside prosthetic valve infections and technical failures of implantation, degeneration and ultimate failure of biological valves arises due to tears the leaflets causing regurgitation or calcification in the leaflets causing stenosis and obstruction to flow through the valve. Calcific stenosis and degenerative changes that lead to tears in the valve leaflets are especially related to areas within the leaflet that are exposed to the greatest stress or strain. Recognition of the role of stress and distributions have led to advances in the design of biological prosthetic valves, specifically the use of flexible supports in place of traditional rigid wire stents. More recently, however, a substantial body of evidence has accumulated for the role for the geometry of the supporting structure to longevity of biologic valves, specifically the presence of dilatations in the wall of the conduit or sinuses of Valsalva. (Redaelli et al. Optimisation of a stentless valve prosthesis based on an analytical parametric model of the aortic valve. The International Journal of Artificial Organs vol. 21 no.3 pp. 161-170). It follows therefore that it may be possible to increase the longevity of tissue engineered valves by incorporating a sinus like geometry to the supporting conduit wall. This poses an immediate problem when the base material is a flat sheet. Whereas the surface of semilunar valve leaflets may largely be mapped or represented by part of the surface of a cylinder, the sinus geometry is closer to that of a ball. In mathematical terms, the leaflets exhibit one axis of curvature and the sinuses at least two. Although it is possible to wrap a flat sheet smoothly over the surface of a cylinder, it is not possible to wrap a flat sheet over a ball without buckling of the textile or taking pleats in it. In fact, modern parametric models of valve leaflets also consider the latter to be rather more complex surfaces than parts of a mere cylinder.

### SUMMARY OF THE INVENTION

In contrast to other tissue engineering techniques, in the subject invention an anatomical element is generated by growing tissue over a scaffold of a predetermined shape in which the scaffold is made from sheets of non-woven porous material, in which the sheets are laminated or joined together through a technique of needling in which needle punching is used to pass a fiber form one sheet into an underlying sheet.

Needlepunching or needlefelting is a method of mechanically interlocking fiber webs by physically repositioning some of the fibers as fiber tufts or bundles from a horizontal to a vertical orientation. The repositioning means is a barbed needle and fiber repositioning is achieved by penetrating the needle into the web in a manner that permits the barbs to carry groups of fibers from one layer to form fiber plug and withdrawing the needle in a manner that permits the fibers to remain in their new position.

The degree of interlocking is dependent primarily on the extent to which the needle penetrates the web, that is the depth of penetration, the needling density, that is the number of penetrations per unit area of fabric, and the number of groups of fibers repositioned per penetration which pertains to the needle design, the number and shape of the barbs on the needle.

What is provided is a system for generating tissue by growing cells in a porous and sometimes biodegradable material, using a needling technique in which a needle punched textile is used for growing any of a variety of anatomical elements, in which delamination is prevented through the use of the needling in those instances in which tissue is grown to form both a series of valve leaflets and a surrounding artery there will be a necessity of somehow bonding a portion of the leaflet to what will become a portion of the artery wall. At these junctures there will of necessity be a layering of a portion of the leaflet with a portion of the artery wall. Delamination at these junctures will cause the leaflets to collapse away from the wall resulting in valve failure.

In one embodiment, the needle punched textile is utilized to form a semi-lunar heart valve in which there is uniform thickness of the members, with the needle punching eliminating the necessity for overlapping and bonding pieces of material in order to make the final shape.

In a preferred embodiment for pediatric use, the textile is made from two different biodegradable non-woven meshes with one sheet made of poly-glycolic acid which biodegrades quickly needled to a sheet of poly L-lactic acid which degrades much more slowly. The result is the ability to produce a substantial amount of tissue conforming to the shape of the textile scaffold before the textile biodegrades.

Pediatric applications include the repair of a wide spectrum congenital defects in anatomical elements or the fabrication of missing elements. The use of forms of multiple axes of curvature over which the sheets are placed and needled permits the fabrication of a scaffold of any particular shape, with the needling permitting the joining together of the edges of the sheets overlying the form without overlapping. The avoidance of overlapping permits fabrication of uniform thickness scaffolds and thus uniform thickness of grown tissue, thus to provide uniform flexibility which grows as the third power of thickness. Needle punching also permits the joining together of sheets as for instance between a valve leaflet and a great artery without the use of sutures and other bonding techniques which oftentimes fail, with the needling technique used to provide support for the commissures over extended areas as opposed to three specific points. The needling technique is used to form a unitary textile sheet without overlapping such that even if the scaffold is initially produced from a number of sheets of material to be joined at their edges, there are no distinguishable layers in the final product.

For adult usage in which the adult anatomical element is full grown in size, then the textile need not be biodegradable but rather may subsist as a scaffold with the newly grown tissue as the size of the element itself will not; grow as would be the case with pediatric subjects. Needling however nonetheless prevents the above mentioned delamination and preserves the uniformity of the thickness of the grown tissue, regardless of the application. This permits symmetrical operation of the various elements, should such be desired.

Because of the low cost involved in tissue engineered anatomical elements the subject technique is not limited to human applications, but may additionally be used to fabricate anatomical elements in other species such as dogs.

In those instances in which tissue is grown to form both a series of valve leaflets and a surrounding artery there will be a necessity of somehow bonding a portion of the leaflet to what will become a portion of the artery wall. At these junctures there will of necessity be a layering of a portion of the leaflet with a portion of the artery wall. Delamination at these junctures will cause the leaflets to collapse away from the wall resulting in valve failure.

The basic problem, therefore, reduce to one of fabricating a non-woven mesh that is macroscopically curved in form rather than flat. It was hypothesized that it would be possible to rearrange fibers in this form using an individual needle in much the same way that layers were interwoven to prevent the above delamination.

In summary, in a system for generating tissue by growing cells in a porous and sometimes biodegradable material, a needle punched textile which serves as a scaffold is used for growing any of a variety of anatomical elements, in which the thickness of areas of the anatomical element and thus its strength can be increased by providing, layers of mesh which are needled together to form a layerless textile and in which delamination is prevented through the use of the needling. In those instances in which tissue is grown to form both a series of valve leaflets and a surrounding artery there will be a necessity of somehow bonding a portion of the leaflet to what will become a portion of the artery wall. At these junctures there will be a layering of a portion of the leaflet with a portion of the artery wall. Delamination at these junctures will cause the leaflets to collapse away from the wall resulting in valve failure, with needling solving the delamination problem. In one embodiment, the needle punched textile is utilized to form a semi-lunar heart valve in which each of the valve leaflets are of uniform thickness since they can be made without overlapping bonded portions, with the needle punching eliminating the necessity for overlapping and bonding pieces of material in order to make the final shape. In a preferred embodiment for pediatric use, the textile is made from two different biodegradable non-woven meshes, without dip coating in a solution of the thermoplastic polymer poly-4-hydroxy butyrate, with one sheet made of poly-glycolic acid which biodegrades quickly needled to a sheet of poly L-lactic acid which degrades much more slowly. The result is the ability to produce a substantial amount of tissue conforming to the shape of the textile scaffold before the textile biodegrades completely. For some adult applications biodegradable meshes are not necessary, thus eliminating the necessity of using two different needled meshes. In one embodiment the needling is done with a single needle which is made to move around the periphery of a mold used in making the scaffold, thus to precisely control the area needled.

### BRIEF DESCRIPTION OF THE DRAWINGS

These and other features of the subject invention will be better understood in conjunction with the Detailed Description in combination with the Drawings, of which:

FIG. 1 is a diagrammatic illustration of a child's heart and lung en bloc showing the absence of any connection between the right ventricle and the pulmonary arteries;

FIG. 2, is a diagrammatic representation of a replacement of the abnormality illustrated in FIG. 1 using a homograft;

FIG. 3 is a diagrammatic illustration of a prior art molding technique having a mold about which is draped mesh so as to produce an inner layer to form the leaflets within a cylindrical outer sheath;

FIG. 4 is a diagrammatic illustration to demonstrate sites of delamination which occur with a scaffold fabricated using the technique of FIG. 1 in which there is delamination between the commissures and the sheath, and between each of the leaflets and the outer sheath;

FIG. 5 is a diagrammatic illustration of the structures formed utilizing the mold of FIG. 3 in which the outer sheath is cylindrical without rounded sinuses;

FIG. 6 is diagrammatic illustration of the sinuses that should exist in the wall of the heart valve of FIG. 5;

FIG. 7 is a diagrammatic illustration of an overlap in forming the outer sheath utilizing the cylindrical mold of FIG. 3, indicating the area of overlap;

FIG. 8 is a diagrammatic illustration of a leaflet formed utilizing the mold of FIG. 3 in which there are areas of overlap in the formation of a leaflet;

FIG. 9 is a diagrammatic illustration of the first step of the subject needling technique utilizing a spherical mold in which sheets of mesh are needled together about the mold and are joined in the needling process so as to create a single valve leaflet-sinus unit;

FIG. 10 is a top view of the trileaflet valve formed from the three units assembled as shown in FIG. 9 through the utilization of a spherical mold and needling, indicating the rounded nature of the sinuses and the union of three valve leaflet-sinus units to form a trileaflet valved structure;

FIG. 11 is a perspective view from the top of a valve formed using needling showing the joining together of various portions of the sheets to provide extra support at the commissures of the valve, with the mesh also permitting the form-ation of rounded sinuses;

FIG. 12 is a perspective view from the top of a valve made in accordance with FIGS. 9, 10 and 11 that has been cut away to show that the needling secures a continuation of the valve leaflet to the outer sheath;

FIG. 13, is a top view of the valve of FIG. 12 showing the tri-leaflet structure and associated sinuses.

FIG. 14 is a flow chart of the steps to grow a tissue engineered heart valve.

FIG. 15 is a diagrammatic illustration of a roller bottle used for cell culture in which the bottle has been provided with culture medium and cells in which the cells grow on the interior wall of the bottle;

FIG. 16 is a diagrammatic illustration of the mounting of a scaffold in a hybridization tube; and,

FIG. 17 is a diagrammatic illustration of the scaffold in a roller bottle for tissue growth on the scaffold.

### DETAILED DESCRIPTION

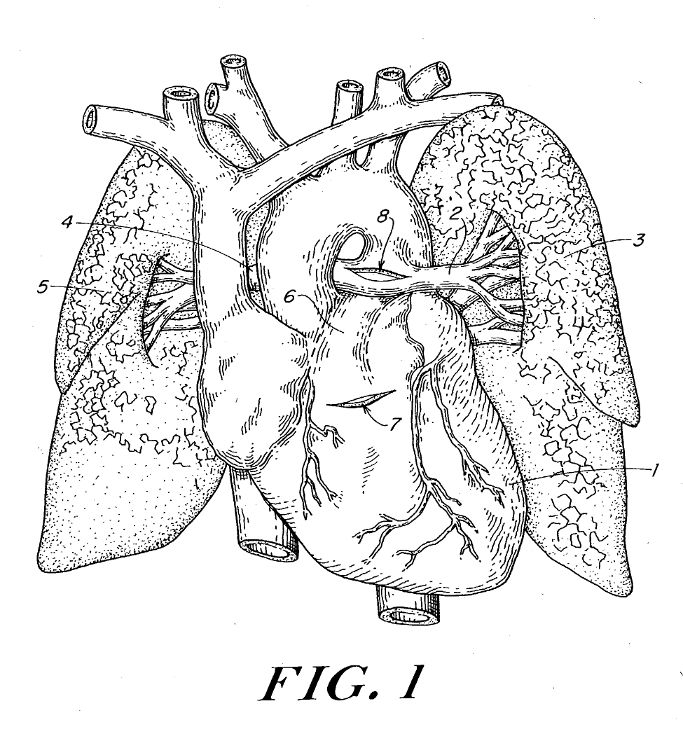

Referring now to FIG. 1, in a child with congenital absence of any connection between the right ventricle of his heart and the pulmonary artery, what can be seen is that for the child's heart a right ventricle 1 normally connects to the confluence of a left pulmonary artery 2 and a right pulmonary 4 connected respectively to a left lung 3 and a right lung 5.

In a normal heart, there is a connection, here shown by dotted line 6, between right ventricle 1 and confluence of pulmonary arteries 2 and 4.

In order to create this connection, an incision 7 is made in the front wall of right ventricle 1, with a similar incision 8 made in the front wall of the confluence of the right and left pulmonaries.

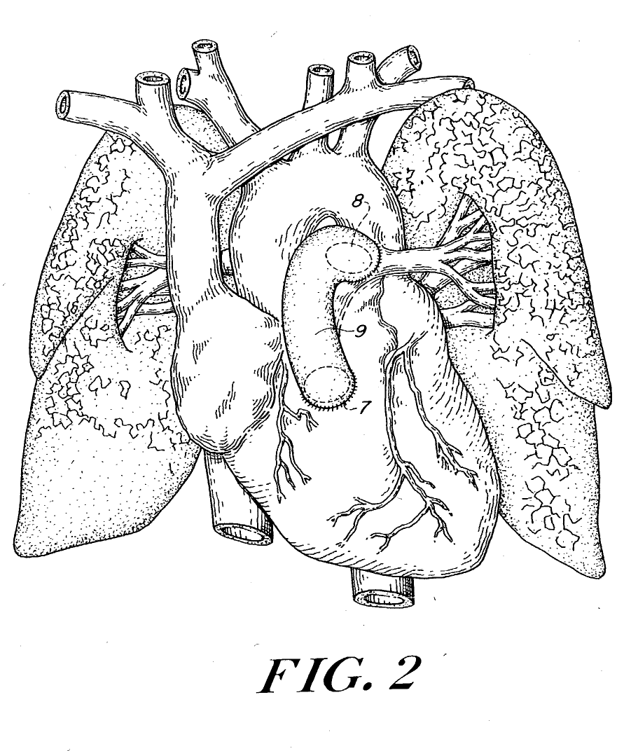

Referring now to FIG. 2, in general practice at present one connects a homograft or Dacron tube between incisions 7 and 8, with the Dacron tube sometimes containing a valve.

For such congenital defective children, the tube and its associated valve establishes blood flow from the right ventricle to the pulmonary arteries and therefore restores the normal physiologic blood flows.

However, long-term outcome from such a procedure using homografts is poor, with a five-year survival rate being an exception. Note, the five-year survival rate for homografts is around 25% for children operated on in their first year of life.

Referring now to FIG. 3, rather than using homografts, in the past mesh was used with tissue engineering to form leaflets. In one prior art process a two part mold is used involving halves 10 and 12 is configured so that the halves have mating cylindrical surfaces and mating portions 14 and 16 shaped to form leaflets of a valve.

In order to form the mesh scaffold for tissue growth, one layer of mesh is draped over the lower mold half 12 and is joined by heat welding of the overlapping join so to create the valve leaflets. The upper mold half 10 is then mated with the lower mold half sandwiching the valve leaflets therebetween.

The first mesh sheet is overwrapped with a second mesh sheet, and joined by heat welding of the overlap to form a cylindrical structure having a cylindrical outer layer 20 as illustrated in FIG. 4. Leaflets 22 comprise an inner layer 24. The inner and outer layers in FIG. 4 are secured together through the utilization of a thermoplastic bonding agent, with the bonded surfaces heat welded together.

As mentioned before, one of the major problems with such a structure is delamination between inner and outer layers, with the delamination 26 at the base of the latter being particularly severe because the commissure is not self supporting. Moreover, walls of a leaflet which are bonded to the outer layer oftentimes come loose due to the flexure of the leaflet and load on the leaflets during the diastolic phase of the cardiac cycle.

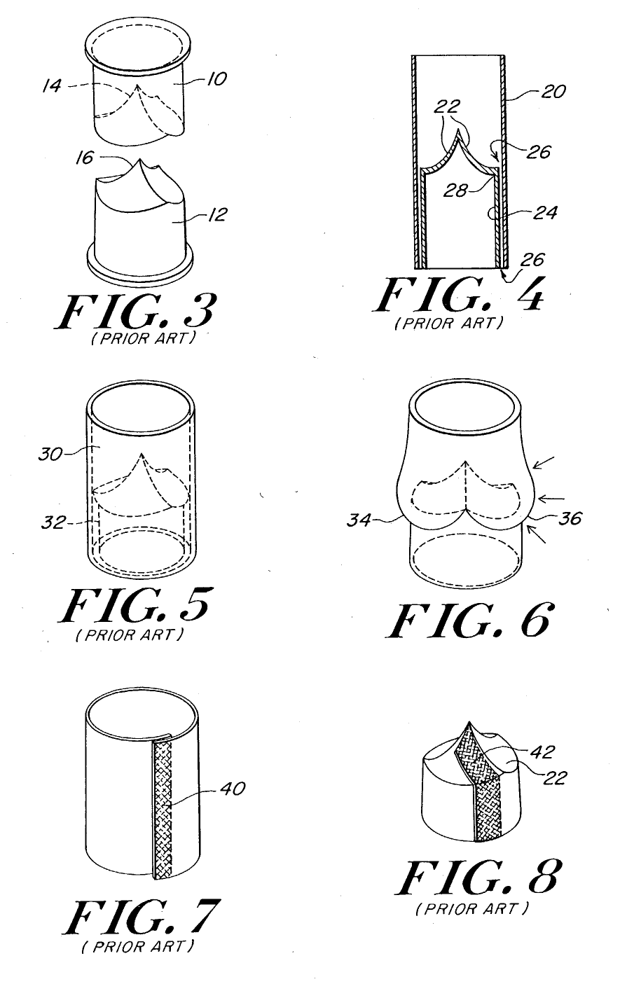

Referring now to FIG. 5, another problem with the prior art molding system is that the walls 30 and 32 of the resulting structures are cylindrical. The result of using the cylindrical mold halves is the inability to provide more complex shapes exhibiting more than ones axis of curvature such as the convex sinuses 34 and 36 of the leaflets of the valve of FIG. 6. Since cylinders have only one axis of curvature, it will be appreciated that a flat mesh is not easily draped over surfaces, with two or more axes of curvature without folds.

Referring to FIG. 7, a still further problem with the utilization of the cylindrical mold of FIG. 3 is that the mesh is formed by overlapping portions which are then welded together. In FIG. 7, the overlap at a welded portion is illustrated at 40 for a conduit or outer sheath. Problems with overlapping materials for a conduit are not as severe as when one is trying to provide a series of leaflets having symmetry and uniformity. Also, welding of the mesh as indicated above provides a line of weakness in the resulting structure.

Referring to FIG. 8, for leaflets 22 when formed through overlapping there is an area 42 of overlap which is both thicker than desired and is therefore much less flexible than the remainder of the leaflet. In addition to being a point of weakness for delamination, the increase in thickness decreases the flexibility of the leaflet to the third power making valves produced in such a manner operate asymmetrically and have the potential to produce eddies in the flow of blood through the valve which are known to promote thrombosis.

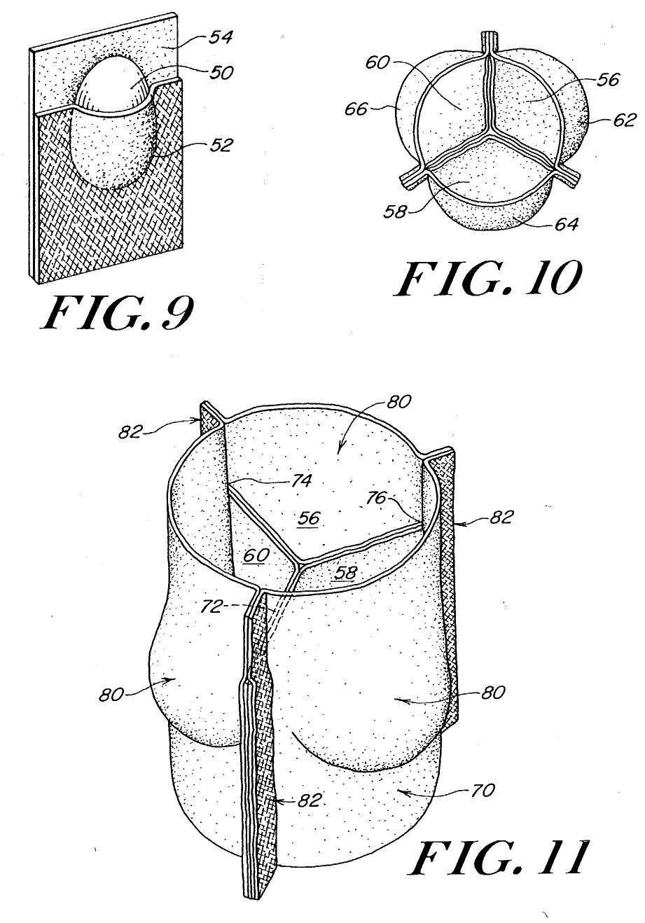

In order to eliminate the above noted problems, in the subject invention a mold 50 as illustrated in FIG. 9 may be spherical. Moreover, the mold itself can be made to have a more complex surface that more exactly replicates the geometry of the normal heart valve due to needling techniques in which a first layer 52 is draped over one side of the mold and is needled to a second layer 54 positioned at the other side of the mold. The needling secures together the portions of these sheets which overlie each other to either side of the mold such that the resulting structure is a unified reinforced mesh which follows the surface of the mold without folds. Note that if the mold is inflatable, then it can be removed after deflation.

The needlepunch process was developed commercially in the late 1800s by William Bywater Ltd., in England. Early applications were focused on coarse animal hair and vegetable fibers for use as carpet underlays and spring padding for mattress and furniture. In the 1920s and 1930s improved needlepunch machinery was introduced. During the late 1950s, needled synthetic-fiber products were introduced to the home furnishings and apparel markets. Several efforts were launched in the 1960s to produce simulated leather with needled fabric as a substrate.

Principal application areas for the 1990s included automotive, apparel components, blankets, carpeting, carpet padding, coating substrates, filtration, furniture, geotextiles, insulation, roofing substrates and wall coverings. Needlepunch production levels were estimated to approach 200 million lb and 725 million yd² in 1990, and 250 million lb and 900 million yd² in 1997.

Non-woven fabrics are made on needlelooms. During the down stroke of the needle beam, each barb carries groups of fibers, corresponding in number to the number of needles and number of barbs (up to 36) per needle, into subsequent web layers a distance corresponding to the penetration depth. During the upstroke of the needle beam, the fibers are released from the barbs and interlocking is established. At the end of the upstroke, the fabric is advanced by the take-up and the cycle is repeated. Needling density is determined by the distance advanced and the number of penetrations per stroke. Needlelooms are produced in widths ranging from several, centimeters to several meters. Needlelooms with low-density boards are used to lightly consolidate webs and are termed pre-needlers or needletackers. Machines with multiple or high-density needle capabilities are often referred to as consolidation or finisher needlelooms. Needles for needlepunch machinery are produced by Foster Needle, Groz-Beckert, Singer, and others.

Note that in the subject case only one needle is used, with the needle being carried in one embodiment on a conventional sewing machine. In operation, the sewing machine needle moves up and down about the periphery of the mold so as to needle together layers of the mesh which exist at the periphery of the mold.

Referring to FIG. 10, in this top view leaflets 56, 58 and 60 can be formed having respective bulbous sinuses 62, 64 and 66 due to the formation of the leaflets about a complex surface, which for simplicity is deemed to be a sphere.

After needling the resulting heart valve is illustrated in FIG. 11 to have an outer conduit wall 70 joined at three commissures 72, 74 and 76, with leaflets 56, 58 and 60 having portions thereof secured to conduit 70 during the needling process.

Because of the utilization of the spherical mold these leaflets and contiguous conduit are provided with sinuses 80 to provide for the function of the valve acutely and for the long term durability of the valve.

Here it can be seen that various portions of the conduit are needled together at 82 to provide for the aforementioned stability of the commissures and to avoid the use of thermosetting resins.

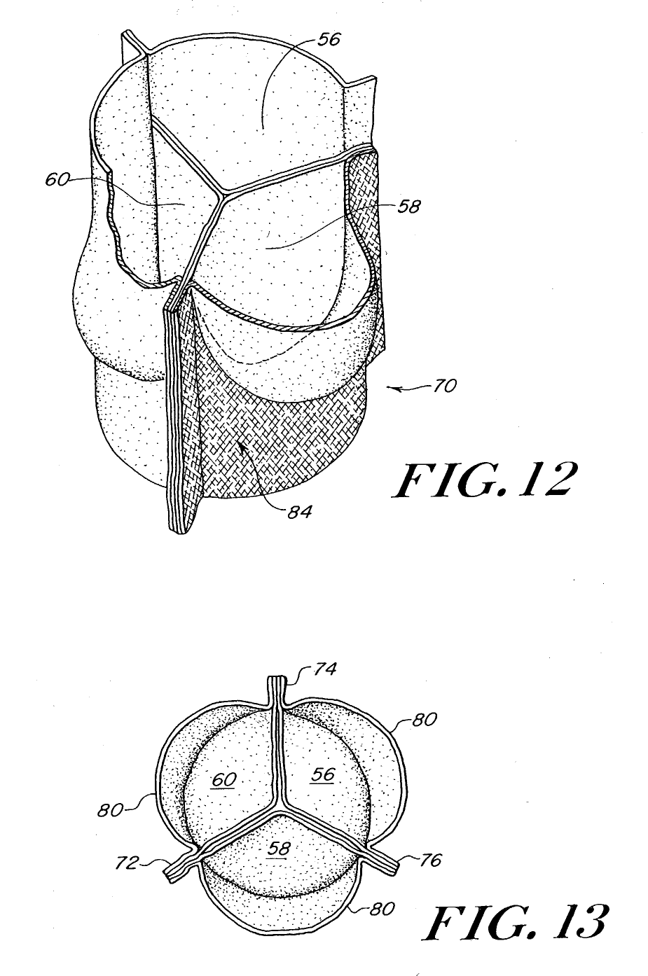

Referring to FIG. 12, not only are the commissures reinforced in their attachment to the conduit wall but with needling, portions of the leaflet are secured to the conduit wall itself. Here it can be seen that leaflet 58 is joined to conduit 70 over the entire area 84, with the needling providing a secure and symmetric support for the leaflet by, the conduit wall.

Symmetry is provided by resolving the structure into three repeating valve/sinus units. Each unit is assembled as a module in its own right with the two layers of the non-woven mesh interwoven by needlepunching in areas that will represent the inflow portion and interleaflet triangles described above. The heart valve scaffold is then assembled by joining three such units along seams that lie external to the conduit wall. These joins are also achieved using the needlepunch technique. This mode of assembly provides equal support to each leaflet at the three commissures and also restores perfect symmetry between the three valve leaflets. In effect, a ‘single piece’ scaffold is created composed entirely of non-woven mesh.

Scaffolds made in this manner provide a uniform structure onto which tissue may be grown in which the tissue is as uniform and symmetric as the underlying scaffold itself. No thermal welding is required which decreases strength and in fact no suturing is required to reinforce parts of the scaffold as was the case with prior scaffolds.

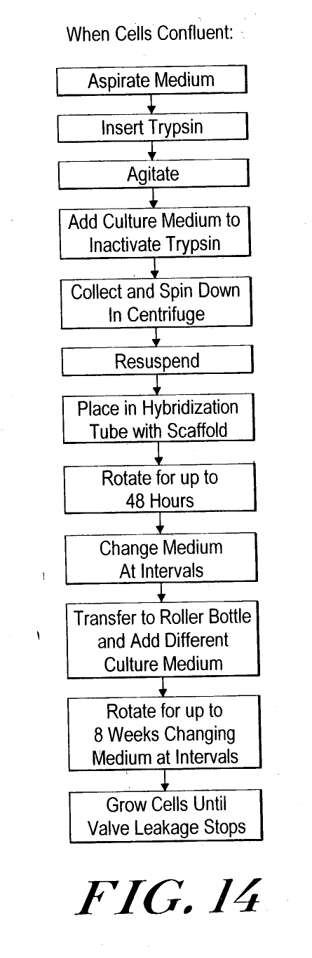

In one embodiment, tissue is, grown on a scaffold through the utilization of roller bottles. In FIG. 15 a roller bottle 109 is provided with culture medium 102 and cells are introduced through orifice 104 so that after a suitable incubation time cells 106 are grown over the interior wall of the roller bottle. When the cells are confluent, then as illustrated by flow chart of FIG. 14, the medium is aspirated and a solution of the enzyme trypsin is injected into the bottle to detach the cells. The injection of the trypsin solution is followed by agitation and the subsequent addition of culture medium to inactivate the trypsin.

At this point the cells are detached and in suspension within the roller bottle. The suspension is then collected and spun down in a centrifuge. The supernatant is aspirated and cells are resuspended in a small volume of fresh culture medium.

Thereafter and as illustrated in FIG. 16 the collected and concentrated suspension of cells is injected into a hybridization tube 110 in which is placed the scaffold 112 and is secured via ‘O’-rings 114 to a segment 116 of tubing in the hybridization tube or to a molded supporting structure.

Referring back to FIG. 16, the hybridization tube is rotated for up to 48 hours with the medium changed every six hours until such time as most of the cells have been delivered onto the scaffold. The culture medium may also be replenished with fresh medium by continuous or intermittent circulation from a much larger reservoir of culture medium. In this system, which incorporates a reservoir, a pump and couplings to each end of the hybridization tube to allow free rotation of the tube about its long axis, the culture medium must be filtered so as to keep the suspended cells within the vicinity of the scaffold so as to permit attachment to the scaffold yet minimize the effective suspending volume of the cells so as to increase the chance of an individual cell encountering the scaffold per unit time.

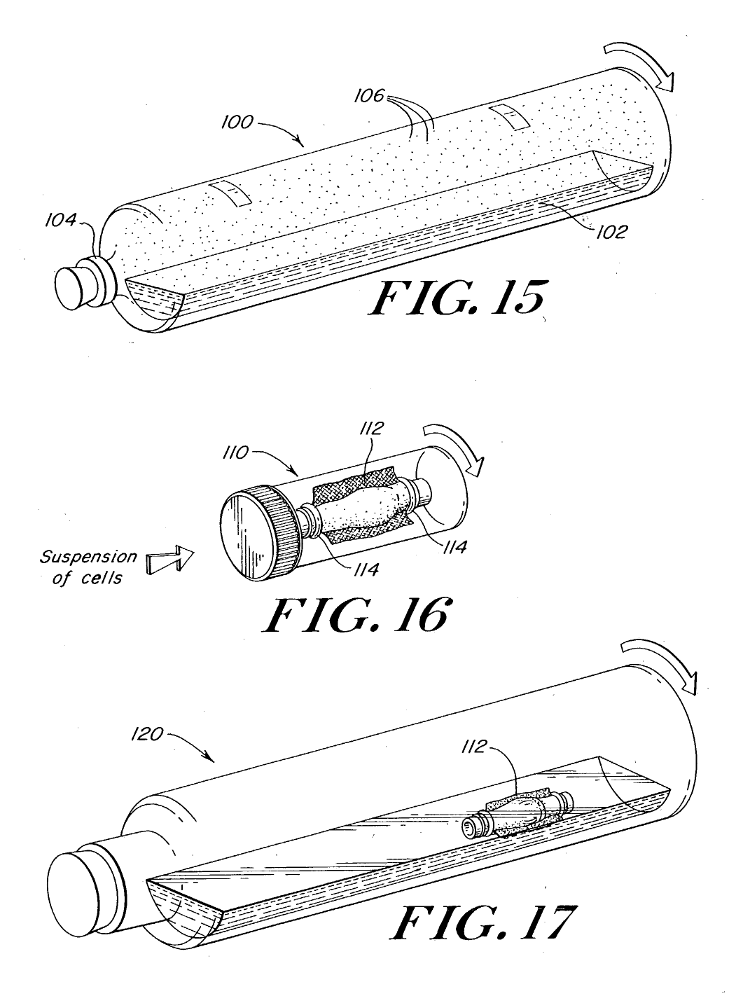

Thereafter and as illustrated in FIG. 17, scaffold 112 is placed in an additional roller bottle 120 which is supplied with a different type of culture medium which has ascorbic acid to promote collagen formation. The formation of collagen is important because tissue is composed of cells and extra-cellular matrix. Collagen is one of the principal components of extra-cellular matrix that provides strength to the nascent tissue.

The roller bottle with scaffold and tissue growing is rotated for up to 8 weeks with the medium changed every 48 hours. The scaffold is placed within the roller bottle in such a manner that when the bottle is placed in an upright position and this valve fills with fluid one can ascertain if there is leakage through the valve. When leakage ceases, there is sufficient build up of tissue on the scaffold such that the entire device may now be used for its intended purpose as a replacement semi-lunar valve. A further step may be incorporated so as to provide endothelial cells on the inner surface of the cell-scaffold construct if so desired.

What will be appreciated is that an anatomical element is produced through the utilization of mesh layers which are needled together over a mold or mandrel so that a unitary scaffold is produced having the desired shape and strength characteristics commensurate with a uniform unitary device. The problems of delamination, inappropriate geometry and non-uniform thicknesses and asymmetries is eliminated through the subject needling technique.

Additionally, the meshes themselves may be biodegradable, with the biodegradation being controllable depending on the composition of fibers used or by what different meshes are needled together.

While the subject invention was described in connection with pediatric appliances, and in which biodegradable scaffolds were thought to be useful so that the tissue engineered appliances could grow with the child ino adulthood, the subject scaffolds may be utilized in adult populations and indeed in non-human species.

More particularly, with respect to pediatric semi-lunar valves, the following techniques are utilized:

Scaffolds were assembled using 1 mm PGA/PLLA non-woven mesh in a ratio of 50:50 PGA:PLLA fibers. For this purpose, the geometry of the valve leaflet-sinus unit was simplified to that of the surface of a sphere of radius Rₗₛ, with geometric continuity between the valve leaflet and its respective sinus of Valsalva.

In this model:

Rₗₛ=1.14Rᵢ (1)

and

Rᵢ=Rₒ (2)

where the Rₒ is the radius of the outflow portion of the valve and Rᵢ the radius at the inflow end of the valve. The geometry of this scaffold may, however, be improved by the use of published mathematical models that seek to optimize all geometric parameters as described by Redaelli A. et al ‘Optimisation of a stentless valve prosthesis based on analytic parametric model of the aortic valve’ The International Journal of Artificial Organs Vol. 21, no. 3, 1998 pp.161-170. Heart valve scaffolds were then mounted on a short section of ¾″ PFA tube manufactured by the Cole Parmer Instrument Company and were secured with rubber ‘O’-rings from the Boston Rubber Company, Boston MA. The scaffolds were inverted and placed in a 140 ml glass hybridization tube. The tube plus scaffold were sterilized by exposure to ethylene oxide.

As to cell harvest and culture, in one embodiment, cells may be harvested from the carotid artery. Briefly, under anesthesia a segment of carotid artery is excised and the artery repaired by end-to-end suture. The piece of artery is transferred to the laboratory in Hank's balanced salt solution and the artery is cut into very small pieces with scissors. The pieces are placed in sterile tissue culture dishes, immersed in Dulbacco's modified Eagle's medium with 10% fetal bovine serum, 5% antibiotic and antimycotic and 20 ng/ml bFGF. After several days, cells are seen to grow out onto the base of the tissue culture dish. The fragments of tissue are then removed and cells expanded into successively larger dishes using standard tissue culture techniques. Cells are finally passaged into 1750 cm² roller bottles. At least eight such bottles confluent with cells are required to make a valve. A variety of other cell types may also be applicable to this technique, including but not limited to cells from the bone marrow, adipose tissue or from peripheral blood.

As to the method of cell delivery onto the scaffolds, approximately 800 million cells are harvested from 1750 cm² roller bottles using a solution of the enzyme trypsin. The cells are resuspended in 25 ml of the Modified Seed Medium, Dulbacco's modified Eagle's medium with 20 mM HEPES buffer with 10% fetal bovine serum, 5% of a solution of antibiotics and antimycotic, 2 ng/ml basic fibroblast growth factor, bFGF, sodium bicarbonate and L-glutamine, and transferred to the hybridization tube. The scaled tube is rotated at 4 cycles per minute at 37° C. in a hybridization oven, namely the HyRoller Hybridization oven from Owl Scientific Inc. At 6 hourly intervals the medium is aspirated, centrifuged, at 1000 rpm for 5 min. and resuspended in fresh medium. The process is repeated for up to 48 hours by which time the residual cell pellet has largely disappeared.

As to culture of the cell-scaffold construct, at the conclusion of the 48 hour cell delivery period, the construct is transferred to a sterile disposable 1750 cm² roller bottle, manufactured by Corning Inc. 300 ml of Construct Culture Medium, Dulbacco's modified Eagle's mediums with L-glutamine, with 20 ng/ml bFGF, 10% fetal bovine serum, 400 mg/l ascorbic acid and additional L-glucose. Residual air is displaced by blowing a 5% CO₂/air mixture (NE Airgas, Salem N.H.) through a 0.2 micrometer filter and 5 ml pipette into the base of the bottle at 12 liters/min for 1 min. thereby providing ac 5% CO₂ enriched atmosphere for optimal buffering and cell growth. The culture bottle is placed on a rack in an incubation cupboard available from Bellco and rotated continuously at 0.2 cycles per minute. Medium and atmosphere are changed at 48 hourly intervals and culture continued for a period of 3-8 weeks.

A preliminary assessment of tissue engineered valves can be made by visual inspection of the surface of the graft through the roller bottle and by gently inverting the bottle so as to fill the valve outflow with fluid and then looking for leakage through the valve. In general valve competency is achieved within 2.5-4 weeks.

When valves are judged to be competent by visual inspection they may be implanted. In order to demonstrate the utility of this method, valves have been implanted into sheep. Briefly, animals are sedated with a cocktail of midazolam and ketamine, intubated and maintained on an isoflurane/air mixture. A central venous line is placed percutaneously in the right internal jugular vein and arterial monitoring established in the left carotid artery by direct exposure. The animal is positioned in the right lateral position and the heart exposed through a left anterior thoracotomy through the 4^{th} rib space. This access provides reasonable exposure to the pulmonary artery and sufficient access to the aorta and right atrium for cannulation. The main PA is mobilized proximally and distally prior to establishing CPB.

Whole blood is drawn from the animal and the tissue engineered semi-lunar heart valve preclotted by gently massaging blood into the wall of the graft, a technique known as ‘preclotting’. A cuff of graft is excised from proximal and distal ends and placed in 4% paraformaldehyde for histological examination.

The bypass circuit comprises an Optima XP oxygenator from Cobe Cardiovascular Inc., Arvada, Colo. with integral 4 liter hard shell reservoir, a ⅜″ arterial limb to the circuit, a 20 Fr. arterial cannula from Bard and a single 40 Fr. cannula positioned in the RA. The circuit is primed with 1200 ml crystalloid and colloid solutions.

The animal is then heparinised and cardiopulmonary bypass established. The temperature is maintained at 37 degrees and the PA opened distally. The native pulmonary valve leaflets are excised and the distal anastomosis performed using a continuous 4/O prolene suture. The native PA is trimmed and proximal anastomosis performed as above whilst the animal is rewarmed to 38° C., the normal ovine core temperature. Suture lines are checked for hemostasis, ventilation is resumed and the animal weaned from CPB. The cannnulae are removed and cannulation sites repaired with 4/0 prolene. Drains are placed in the pericardium and left thoracic cavity and connected to, underwater seal. The chest is closed in layers. The animal is kept sedated with a cocktail of fentanyl and midazolam whilst vitals signs and chest drainage are monitored. When stable the animal is delined and returned to its pen. Aspirin and antibiotics are administered during the postoperative period.

As to functional assessment of tissue engineered valve in situ an echocardiogram is performed at the conclusion of the surgery prior to closing the chest to assess performance. The following parameters are measured and recorded: effective orifice area, peak systolic gradient across the valve, estimation of regurgitation and qualitative assessment of leaflet motion. The valve leaflets open and close symmetrically. There is minimal pressure gradient across the valve, less than 15 mmHg, and there is trivial regurgitation. The effective orifice area is of the order of 2.1 cm² for a 21 mm diameter valve consistent with its function as a substitute pulmonary valve Histological examination of the tissue engineered valve after preclotting but prior to implantation has been performed and demonstrates a density of tissue over the entire construct that is consistent with its proposed function as a substitute pulmonary valve.

A tissue engineered valve made and implanted by the method described has been examined on the second postoperative day. The valve has been photographed in situ and then explanted together with a cuff of normal pulmonary artery.

In summary in the subject invention, the first step was to show that separation between layers may be prevented by the provision of through thickness members over those areas of the scaffold where two layers of textile are apposed. This was achieved by needle punching. The next step was to recognize that it is not possible to assemble a structure with the complex series of curves and forms that characterize a normal semi-lunar heart valve from flat sheets alone. Whilst it is clearly possible to drape a flat sheet smoothly over a surface with one axis of curvature, such as a cylinder, it is not possible to drape the same sheet over a surface with two or more axes of curvature, such as a sphere, without folds appearing in the textile. However, by needle punching around the periphery of such a shape, it is possible to reorient the fibers contained therein and to create a textile with a smooth curved geometry from one that was initially flat. Finally, the normal heart valve geometry was resolved along its lines of symmetry into three identical repeating valve-sinus units. A method for assembling each unit was devised using the above principals and the units joined along a seam that was brought out external to the conduit. This method provides a heart valve scaffold that recapitulates the complex geometry of the native structure, with curved valve leaflets and the multiply curved sinuses of Valsalva. Furthermore, the external seam provides equal support to each of the heart valve leaflets and distributes stress away from the three commissures.

Having now described a few embodiments of the invention and some modifications and variations thereto, it should be apparent to those skilled in the art that the foregoing is merely illustrative and not limiting, having been presented by the way of example only. Numerous modifications and other embodiments are within the scope of one, of ordinary skill in the art and are contemplated as falling within the scope of the invention as limited only by the appended claims and equivalents thereto.

What is claimed is:

1. A system for generating tissue using a scaffold at least partially made up of needle punched textile.

2. The system of claim 1, wherein said needle punched textile includes biodegradable textile.

3. The system of claim 1, wherein said textile is a non-woven textile.

4. The system of claim 1, wherein the textile includes polygycolic acid.

5. The system of claim 1, wherein the textile includes poly L-lactic acid.

6. The system of claim 1, wherein the density of the textile is between 50 and 100 milligrams per cubic centimeter.

7. A system for varying the biodegradability of a scaffold used in tissue engineering, comprising:
a number of different layers of mesh each having a different biodegradability, said layers being needle punched together.

8. The system of claim 7, wherein the layers include polymer and wherein the residual mass of polymer over time is diminished.

9. A method of forming a scaffold for tissue generation, comprising:
providing a number of layers of non-woven mesh and a mold; and,
needling around the periphery of the mold so as to join portions of the layers at the periphery of the mold.

10. The method of claim 9, wherein the geometry of the mold approximates the normal geometry of an anatomical element.

11. The method of claim 9, wherein the needling is done with a single needle.

12. The method of claim 9, wherein the scaffold is used for forming a heart valve.

13. A method of forming a uniform thickness of scaffold for growing tissue to form anatomical parts, comprising:
making a least a portion of the scaffold from two layers of non-woven mesh; and,
needling the layers together to form a uniform thickness layerless textile.

14. A method of culturing cell-scaffold constructs for the production of substitute anatomical elements, comprising the steps of:
putting cells onto the scaffold using a hybridization tube until some cells attach to the scaffold;
placing the scaffold into a roller bottle;
placing culture medium in a roller bottle; and,
rotating the roller bottle in an incubator to proliferate the cells and for the cells to lay down extra cellular matrix.

15. The method of claim 14, and further including injecting a gas into the roller bottle to buffer the pH of the culture medium to physiological levels.

16. The method of claim 14, wherein the anatomical element includes a heart valve.

17. The method of claim 14, wherein the culture medium is Dulbacco's modified Eagle's medium with additional basic fibroblast growth factor and ascorbic acid.

18. The method of claim 17, wherein the concentration of the basic fibroblast growth factor is below that which is toxic to the cells.

19. The method of claim 17, wherein the concentration of ascorbic acid is below that which is toxic to the cells.

20. The method of claim 14, wherein the incubator is set between 30° C. and 40° C.

21. The method of claim 14, and further including adding glucose to the bottle to increase osmolality.

22. The method of claim 14, and further including adding bovine serum for additional growth factors.

23. The method of claim 14, and further including adding at least one antibiotic.

24. The method of claim 14, wherein the culture medium is serum-free.

25. The method of claim 14, wherein the cells are stem cells.

26. The method of claim 14, wherein the cells are somatic cells.

27. The method of claim 14, wherein the: cells are mesenchymal stem cells.

28. The method of claim 14, wherein the cells are those which are capable of differentiating into interstitial cells of the type the anatomical element is designed to replace.

29. A method for constructing a synthetic heart valve, comprising the steps of:
forming a tissue engineered heart valve using needle punched textile layers; and
implanting the valve.

30. The method of claim 29, wherein the heart valve includes a biodegradable scaffold on which cells are grown.

31. The method of claim 29, wherein the heart valve includes a scaffold in which cells are grown.

32. The method of claim 29, wherein the valve is a tri-leaflet valve.

33. The method of claim 29, wherein the valve is a bi-leaflet valve.

34. An implantable pediatric heart valve comprising:
a biodegradable scaffold defining a valve geometry and including layers of textile mesh needle punched together in selected areas; and,
cells grown on said scaffold to form said valve, said-scaffold degrading over time to leave in place a valve made of cells and an extra cellular matrix.
